(19) United States
(12) Patent Application Publication
(10) Pub. No.: US 2019/0031842 A1
(43) Pub. Date: Jan. 31, 2019

(54) Molded Article, Production Method for Same, and Method for Improving Degree of Crystallization of Molded Article

(71) Applicants: Riken, Saitama (JP); Spiber Inc., Yamagata (JP); Kojima Industries Corporation, Aichi (JP)

(72) Inventors: Keiji Numata, Saitama (JP); Kana Ishida, Yamagata (JP); Hironori Yamamoto, Aichi (JP)

(73) Assignees: Riken, Saitama (JP); Spiber Inc., Yamagata (JP); Kojima Industries Corporation, Aichi (JP)

(21) Appl. No.: 16/073,069

(22) PCT Filed: Jan. 27, 2017
(86) PCT No.: PCT/JP2017/003044

§ 371 Date: Jul. 26, 2018

(30) Foreign Application Priority Data
Jan. 29, 2016 (JP) 2016-015593

(51) Int. Cl. C08J 5/18; C07K 14/435

(52) U.S. Cl. CPC C08J 5/18; C08J 2489/00; C07K 14/43518; C07K 14/43536

(57) ABSTRACT

The present invention provides, in one aspect, a method for producing a molded article, the method comprising exposing a molded article precursor comprising a protein to an environment with a relative humidity of 80% or more to obtain the molded article.

### TECHNICAL FIELD

The present invention relates to a molded article and a method for producing the same, as well as a method for improving the degree of crystallization of the molded article.

### BACKGROUND ART

Due to the recent rise in awareness of environment preservation, consideration of alternative materials for materials derived from petroleum has been promoted. Proteins, which are excellent in twits of strength etc., are considered as candidates for such alternative materials. Proteins can also be applied to molded articles such as films and fibers which, conventionally, have mainly been made of materials derived from petroleum. For example, Patent Literature 1 discloses a biodegradable molded article comprising a protein, a plasticizer, a degradation retarder and/or a water resistance-imparting agent.

### CITATION LIST

### Patent Literature

Patent Literature 1: Japanese Unexamined Patent Publication No. H8-73613

### SUMMARY OF INVENTION

### Technical Problem

An object of the present invention is to provide a molded article having a high degree of crystallization, and a method for producing the same.

### Solution to Problem

The present inventors have examined molded articles containing a protein, and consequently have found that the degree of crystallization of the molded articles is improved by exposing the molded articles to environments with a high relative humidity, although the mechanism thereof, and the structure and characteristics of the molded articles after exposure are not clear. The present inventors assume that molded articles having excellent stress, elastic modulus, etc., can be obtained by improving the degree of crystallization of the molded articles.

The present invention provides, in one aspect, a method for producing a molded article, comprising exposing a molded article precursor comprising a protein to an environment with a relative humidity of 80% or more to obtain the molded article.

The present invention provides, in another aspect, a molded article comprising a protein having an exposure history to an environment with a relative humidity of 80% or more.

The present invention provides, in another aspect, a method for improving a degree of crystallization of a molded article comprising a protein, comprising exposing the molded article to an environment with a relative humidity of 80% or more.

### Advantageous Effects of Invention

The present invention can provide a molded article having a high degree of crystallization, and a method for producing the same.

### BRIEF DESCRIPTION OF DRAWINGS

FIG. 1 are schematic diagrams for explaining a method for exposing a sample to a saturated salt solution environment.

FIG. 2 is a graph showing the results of wide-angle X-ray scattering measurements of silkworm films.

FIG. 3 is a graph showing the relationship between the relative humidity and the degree of crystallization investigated on silkworm films.

FIG. 4 is a graph showing the results of wide-angle X-ray scattering measurements of spider silk fibroin films.

FIG. 5 is a graph showing the relationship between the relative humidity and the degree of crystallization investigated on spider silk fibroin films.

### DESCRIPTION OF EMBODIMENTS

Embodiments of the present invention are described in detail below.

The method for producing a molded article according to one embodiment comprises at least an exposure step of exposing a molded article precursor containing a protein to an environment with a relative humidity of 80% or more.

The molded article and molded article precursor according to the present embodiment (hereinafter, these are also simply referred to in a collective manner as “the molded article”) contain a protein, preferably as a main component. The content of protein based on the entire molded article is not particularly limited. The molded article may contain impurities etc. other than the protein which is the main component. The type of protein is also not particularly limited; for example, a structural protein or a protein derived from the structural protein can be used. Structural protein is a protein that forms or maintains structures, forms, etc., in the living body. Examples of structural protein include fibroin, keratin, collagen, elastin, and resilin.

The structural protein may contain one or more members selected from the group consisting of fibroin and keratin. Fibroin may be, for example, one or more members selected from the group consisting of silk fibroin, spider silk fibroin, and hornet silk fibroin. The structural protein may be silk fibroin, spider silk fibroin, or a combination thereof. When silk fibroin and spider silk fibroin are used in combination, the ratio of silk fibroin may be, for example, 40 parts by mass or less, 30 parts by mass or less, or 10 parts by mass or less, based on 100 parts by mass of spider silk fibroin.

Silk is a fiber obtained from cocoons made by silkworms, which are larvae of Bombyx mori. In general, one cocoon fiber consists of two silk fibroins and glue (sericin) covering the silk fibroins from the outside. Each silk fibroin is composed of many fibrils. The silk fibroins are covered with four layers of sericin. Practically, silk filaments obtained by removing sericin on the outside by dissolving it by purification are used for clothing applications. General silk has a specific gravity of 1.33, an average fineness of 3.3 decitex, and a fiber length of about 1300 to 1500 m. The silk fibroin is obtained using cocoons of natural or domestic silkworms, or used or disposed silk clothes as raw materials.

The silk fibroin may be sericin-removed silk fibroin, sericin-unremoved silk fibroin, or a combination thereof. Sericin-removed silk fibroin is obtained by purifying silk fibroin by removing sericin covering the silk fibroin, other fats, etc. The silk fibroin purified in this manner is preferably used as a freeze-dried powder. Sericin-unremoved silk fibroin is unpurified silk fibroin from which sericin etc. are not removed.

Hornet silk fibroin is a protein produced by bee larvae, and may contain a polypeptide selected from the group consisting of natural hornet silk proteins and polypeptides derived from natural hornet silk proteins.

Spider silk fibroin may contain a spider silk polypeptide selected from the group consisting of natural spider silk proteins and polypeptides derived from natural spider silk proteins.

Examples of natural spider silk proteins include spigot dragline proteins, spiral line proteins, and minor ampullate gland proteins. The spigot dragline has a repetitive region composed of crystalline and amorphous regions, and is thus assumed to have high stress and stretchability. The spider spiral line does not have crystalline regions, but have a repetitive region composed of amorphous regions. On the other hand, the spiral line has high stretchability, although its stress is inferior to that of the spigot dragline. This is considered to be because most part of the spiral line is composed of amorphous regions.

Spigot dragline proteins are produced in the major ampullate glands of spiders, and characteristically have excellent toughness. Examples of Spigot dragline proteins include major ampullate spidroins MaSp1 and MaSp2 derived from Nephila clavipes, and ADF3 and ADF4 derived from Araneus diadematus. ADF3 is one of the two primary dragline proteins of Araneus diadematus. Polypeptides derived from natural spider silk proteins may be polypeptides derived from these dragline proteins. Polypeptides derived from ADF3 can be relatively easily synthesized, and have excellent characteristics in terms of high elongation and toughness.

Spiral line proteins are produced in the flagelliform glands of spiders. Examples of spiral line proteins include flagelliform silk proteins derived from Nephila clavipes.

Polypeptides derived from natural spider silk proteins may be recombinant spider silk proteins. Examples of recombinant spider silk proteins include variants, analogs, derivatives, or the like of natural spider silk proteins. Preferable examples of such polypeptides include recombinant spider silk proteins of spigot dragline proteins (hereinafter also referred to as “polypeptides derived from spigot dragline proteins).

Examples of proteins derived from the spigot dragline and proteins derived from silkworm silk, which are fibroin-like proteins, include proteins containing a domain sequence represented by the formula 1: [(A)ₙ motif-REP1]ₘ, (wherein, in the formula 1, (A)ₙ motif represents an amino acid sequence composed of 4 to 20 amino acid residues, and the number of alanine residues relative to the total number of amino acid residues in (A)ₙ motif is 80% or more; REP1 represents an amino acid sequence composed of 10 to 200 amino acid residues; m represents an integer of 8 to 300; a plurality of (A)ₙ motifs may be the same or different amino acid sequences; and a plurality of REP1 may be the same or different amino acid sequences). Specific examples thereof include proteins comprising the amino acid sequence represented by SEQ ID NO: 1.

Examples of proteins derived from spiral line proteins include proteins containing a domain sequence represented by the formula 2: [REP2]ₒ (wherein, in the formula 2, REP2 represents an amino acid sequence composed of Gly-Pro-Gly-Gly-X; X represents at least one amino acid selected from the group consisting of alanine (Ala), serine (Ser), tyrosine (Tyr), and valine (Val); and o represents an integer of 8 to 300). Specific examples thereof include proteins comprising the amino acid sequence represented by SEQ ID NO: 2. The amino acid sequence represented by SEQ ID NO: 2 is obtained by bonding an amino acid sequence (referred to as the PR1 sequence) from the 1220th residue to the 1659th residue from the N-terminal corresponding to the repeated part and motif of a partial sequence of flagelliform silk protein of Nephila clavipes obtained from the NCBI database (NCBI Accession Number: AAF36090, GI: 7106224) to a C-terminal amino-acid sequence from the 816th residue to the 907th residue from the C-terminal of a partial sequence of flagelliform silk protein of Nephila clavipes obtained from the NCBI database (NCBI Accession Number: AAC38847, GI: 2833649); and adding the amino acid sequence represented by SEQ ID NO: 7 (tag sequence and hinge sequence) to the N-terminal of the bound sequence.

Examples of proteins derived from collagen include proteins containing a domain sequence represented by the formula 3: [REP3]ₚ (wherein, in the formula 3, p represents an integer of 5 to 300; REP3 represents an amino acid sequence composed of Gly-X-Y; X and Y represent any amino acid residues other than Gly; and a plurality of REP3 may be the same or different amino acid sequences). Specific examples thereof include proteins comprising the amino acid sequence represented by SEQ ID NO: 3. The amino acid sequence represented by SEQ ID NO: 3 is obtained by adding the amino acid sequence represented by SEQ ID NO: 7 (tag sequence and hinge sequence) to the N-terminal of an amino acid sequence from the 301st residue to the 540th residue corresponding to the repeated part and motif of a partial sequence of human collagen type 4 obtained from the NCBI database (NCBI Genebank Accession Number: CAA56335.1, GI: 3702452).

Examples of proteins derived from resilin include proteins containing a domain sequence represented by the formula 4: [REP4]_{q} (wherein, in the formula 4, q represents an integer of 4 to 300; REP4 represents an amino acid sequence composed of Ser-J-J-Tyr-Gly-U-Pro; J represents any amino acid residue, and particularly preferably an amino acid residue selected from the group consisting of Asp, Ser, and Thr; U represents any amino acid residue, and particularly preferably an amino acid residue selected from the group consisting of Pro, Ala, Thr, and Ser; and a plurality of REP4 may be the same or different amino acid sequences). Specific examples thereof include proteins comprising the amino acid sequence represented by SEQ ID NO: 4. The amino acid sequence represented by SEQ ID NO: 4 is obtained by adding the amino acid sequence represented by SEQ ID NO: 7 (tag sequence and hinge sequence) to the N-terminal of an amino acid sequence from the 19th residue to the 321st residue of a sequence obtained by substituting the 87th residue Thr with Ser, and also substituting the 95th residue Asn with Asp in the amino acid sequence of resilin (NCBI Genebank Accession Number: NP 611157, Gl: 24654243).

Examples of proteins derived from elastin include proteins having amino acid sequences such as those of NCBI Genebank

Accession Numbers: AAC98395 (human), 147076 (sheep), and NP786966 (cow). Specific examples thereof include proteins comprising the amino acid sequence represented by SEQ ID NO: 5. The amino acid sequence represented by SEQ ID NO: 5 is obtained by adding the amino acid sequence represented by SEQ ID NO: 7 (tag sequence and hinge sequence) to the N-terminal of an amino acid sequence from the 121st residue to the 390th residue of the amino acid sequence of NCBI Genebank Accession Number: AAC98395.

Examples of proteins derived from keratin include type I keratin of Capra hircus, etc. Specific examples thereof include proteins comprising the amino acid sequence represented by SEQ ID NO: 6 (amino acid sequence of NCBI Genebank Accession Number: ACY3 0466).

The abovementioned structural proteins and proteins derived from the structural proteins can be used singly or in combination of two or more.

The protein contained in the protein molded article and the protein molded article precursor as a main component can be produced by, for example, expressing a nucleic acid encoding the protein using a host transformed with an expression vector having one or more regulatory sequences operably linked to the sequence of the nucleic acid.

The method for producing the nucleic acid encoding the protein contained in the protein molded article and the protein molded article precursor as a main component is not particularly limited. For example, the nucleic acid can be produced by a method of amplifying a gene by polymerase chain reaction (PCR) etc. for cloning, or by a chemical synthesis method, both using a gene encoding a natural structural protein. The method for chemically synthesizing the nucleic acid is also not particularly limited. For example, a gene can be chemically synthesized by linking oligonucleotides automatically synthesized using AKTA oligopilot plus 10/100 (produced by GE Healthcare Japan), etc., by PCR or the like based on amino acid sequence information of structural proteins obtained from the NCBI web database, etc. Under this circumstance, in order to facilitate the purification and/or confirmation of the protein, it is possible to synthesize a nucleic acid encoding a protein comprising an amino acid sequence obtained by adding an amino acid sequence composed of a start codon and His 10 tags to the N-terminal of the abovementioned amino acid sequence.

The regulatory sequence is a sequence that regulates the expression of a recombinant protein in a host (e.g., a promoter, an enhancer, a ribosome-binding sequence, a transcriptional termination sequence, etc.). The regulatory sequence can be suitably selected depending on the type of host. The promoter may be an inducible promoter that functions in host cells, and can induce the expression of a target protein. The inducible promoter is a promoter that can control transfer by the presence of an inductor (an expression-inducing agent), the absence of repressor molecules, or physical factors such as increase or decrease in temperature, osmotic pressure, or pH value.

The type of expression vector can be suitably selected from plasmid vectors, viral vectors, cosmid vectors, fosmid vectors, artificial chromosome vectors, etc., depending on the type of host. Preferable examples of the expression vector include those that are capable of self-replicating in host cells or of being introduced into the chromosome of the host, and that contain a promoter in a position to which a nucleic acid encoding a target protein can be transferred.

As the host, any of prokaryotes, and eukaryotes such as yeast, filamentous fungi, insect cells, animal cells, and plant cells, can be suitably used.

Preferable examples of prokaryotic hosts include bacteria belonging to the genera Escherichia, Brevibacillus, Serratia, Bacillus, Microbacterium, Brevibacterium, Corynebacterium, Pseudomonas, and the like. Examples of microorganisms belonging to the genus Escherichia include Escherichia coli, etc. Examples of microorganisms belonging to the genus Brevibacillus include Brevibacillus agri, etc. Examples of microorganisms belonging to the genus Serratia include Serratia liquefaciens, etc. Examples of microorganisms belonging to the genus Bacillus include Bacillus subtilis, etc. Examples of microorganisms belonging to the genus Microbacterium include Microbacterium ammoniaphilum, etc. Examples of microorganisms belonging to the genus Brevibacterium include Brevibacterium divaricatum, etc. Examples of microorganisms belonging to the genus Corynebacterium include Corynebacterium ammoniagenes, etc. Examples of microorganisms belonging to the genus Pseudomonas include Pseudomonas putida, etc.

When a prokaryotic host is used, examples of the vector for introducing a nucleic acid encoding a target protein include pBTrp2 (produced by Boehringer Mannheim), pGEX (produced by Pharmacia), pUC18, pBluescript II, pSupex, pET22b, pCold, pUB110, and pNCO2 (Japanese Unexamined Patent Publication No. 2002-238569), and the like.

Examples of eukaryotic hosts include yeast and filamentous fungi (mold etc.). Examples of yeast include yeast belonging to the genera Saccharomyces, Pichia, Schizosaccharomyces, and the like. Examples of filamentous fungi include filamentous fungi belonging to the genera Aspergillus, Penicillium, Trichoderma, and the like.

When a eukaryotic host is used, examples of the vector for introducing a nucleic acid encoding a target protein include YEP13 (ATCC37115), YEp24 (ATCC37051), and the like. The method for introducing an expression vector into the abovementioned host cells may be any method as long as it is a method for introducing DNA into the host cells. Examples of the method include a method using calcium ions (Proc. Natl. Acad. Sci. USA, 69, 2110 (1972)), an electroporation method, a spheroplast method, a protoplast method, a lithium acetate method, a competent method, and the like.

The method for expressing the nucleic acid by a host transformed with an expression vector may be direct expression. In addition, secretory production, fusion protein expression, etc., can be performed according to the method described in the 2nd Edition of Molecular Cloning.

The protein can be produced by, for example, culturing a host transformed with an expression vector in a culture medium, allowing the production and accumulation of the protein in the culture medium, and harvesting the protein from the culture medium. The method for culturing the host in the culture medium can be performed according to a process generally used for host culture.

When the host is a eukaryote such as Escherichia coli or a prokaryote such as yeast, the culture medium may be a natural medium or a synthetic medium as long as it contains a carbon source, a nitrogen source, an inorganic salt, etc. that can be assimilated by the host and the host can be efficiently cultured.

The carbon source may be one that can be assimilated by the abovementioned transformed microorganisms. Examples thereof include glucose, fructose, sucrose, and molasses containing them; carbohydrates such as starch and starch hydrolysates; organic acids such as acetic acid and propionic acid; and alcohols such as ethanol and propanol. Examples of the nitrogen source include ammonia, ammonium salts of inorganic acids or organic acids such as ammonium chloride, ammonium sulfate, ammonium acetate, and ammonium phosphate; other nitrogen-containing compounds; peptone, meat extract, yeast extract, corn steep liquor, casein hydrolysate, soybean cake, soybean cake hydrolyzate, various fermentative bacteria and digests thereof. Usable examples of inorganic salts include monopotassium phosphate, dipotassium phosphate, magnesium phosphate, magnesium sulfate, sodium chloride, ferrous sulfate, manganese sulfate, copper sulfate, and calcium carbonate.

Prokaryotes such as Escherichia coli or eukaryotes such as yeast can be cultured under aerobic conditions by shaking culture or aeration agitation submerged culture, for example. The culture temperature is 15 to 40° C., for example. The culture time is generally 16 hours to 7 days. The pH of the culture medium during culture is preferably maintained at 3.0 to 9.0. The pH of the culture medium can be adjusted using inorganic acids, organic acids, alkali solutions, urea, calcium carbonate, ammonia, etc.

Moreover, antibiotics, such as ampicillin and tetracycline, may be added to the culture medium during culture, if necessary. When a microorganism transformed with an expression vector using an inducible promoter as a promoter is cultured, an inducer may be added to the medium, if necessary. For example, when a microorganism transformed with an expression vector using a lac promoter is cultured, isopropyl-β-D-thiogalactopyranoside or the like may be added to the medium; and when a microorganism transformed with an expression vector using a trp promoter is cultured, indole acrylate or the like may be added to the medium.

The expressed protein can be isolated and purified by a generally used method. For example, when the protein is expressed in a soluble state in the cells, the host cells are collected by centrifugal separation after completion of the culture, and suspended in a water-based buffer. Then, the host cells are disrupted by an ultrasonic disruption machine, a French press, a Manton-Gaulin homogenizer, a Dyno-Mill, etc., and a cell-free extract is obtained. The cell-free extract is centrifuged to obtain a supernatant, from which a purified preparation can be obtained by methods generally used for the isolation and purification of proteins, all of which can be used singly or in combination, such as a solvent extraction method, a salting-out method using ammonium sulfate etc., a desalination method, a precipitation method using an organic solvent, an anion-exchange chromatography method using resins such as diethylaminoethyl (DEAE)-sepharose and DIMON HPA-75 (produced by Mitsubishi Kasei Corp.), a cation-exchange chromatography method using resins such as S-Sepharose FF (produced by Pharmacia), a hydrophobic chromatography method using resins such as butyl sepharose and phenyl sepharose, a gel-filtration method using molecular sieving, an affinity chromatography method, a chromatofocusing method, and an electrophoresis method such as isoelectric focusing.

Moreover, when the protein is expressed while forming insoluble fractions in the cells, the insoluble fractions of the protein are collected as precipitation fractions by similarly collecting the host cells, followed by disruption and centrifugal separation. The collected insoluble fractions of the protein can be solubilized by a protein modifier. After this operation, a purified preparation of the protein can be obtained by the same isolation and purification method as described above. When the protein is secreted outside the cells, the protein can be collected from the culture supernatant. More specifically, the culture is treated by centrifugal separation or like method to obtain a culture supernatant, and a purified preparation can be obtained from the culture supernatant by the same isolation and purification method as described above.

The molecular weight of the protein or polypeptide may be 500 kDa or less, 300 kDa or less, 200 kDa or less, or 100 kDa or less, and may be 10 kDa or more, in terms of productivity in the production of recombinant proteins using a microorganism, such as Escherichia coli, as a host. The molecular weight of the protein or polypeptide may be further increased by crosslinking those having molecular weights within the above range with each other.

The structural protein, such as silk fibroin or spider silk fibroin, may be used in combination with other proteins. Examples of other proteins include collagen, soybean proteins, casein, keratin, and whey proteins. The physical properties derived from proteins can be adjusted by the combined use of the structural protein with other proteins. The ratio of other proteins when used in combination may be, for example, 40 parts by mass or less, 30 parts by mass or less, or 10 parts by mass or less, based on 100 parts by mass of the structural protein.

The molded article according to the present embodiment is not particularly limited, and may be a film, fiber, foam, resin plate, or the like. The film is obtained, for example, by a method comprising forming a membrane of a protein solution containing a protein and a solvent, and removing the solvent from the formed membrane. The fiber is obtained, for example, by a method comprising spinning a protein solution containing a protein and a solvent, and removing the solvent from the spun protein solution. That is, the method for producing a molded article according to the present embodiment may further comprise, before the exposure step, for example, a molding step of molding a molded article precursor from a protein solution containing a protein and a solvent.

The solvent used in the molding step may be, for example, a polar solvent. The polar solvent may include, for example, one or more solvents selected from the group consisting of water, dimethylsulfoxide (DMSO), dimethylformamide (DMF), hexafluoroacetone (HFA), and hexafluoroisopropanol (HFIP). The polar solvent may be dimethylsulfoxide alone or a mixed solvent of dimethylsulfoxide and water in terms of obtaining a higher concentration solution, and may be water in terms of reducing adverse effects on the environment.

The content of protein in the protein solution may be 15 mass % or more, 30 mass % or more, 40 mass % or more, or 50 mass % or more, based on the total mass of the protein solution. The content of protein may be 70 mass % or less, 65 mass % or less, or 60 mass % or less, based on the total mass of the protein solution, in teiins of the production efficiency of the protein solution.

The protein solution may further contain one or more inorganic salts, in addition to the protein and the solvent. Examples of inorganic salts include inorganic salts composed of Lewis acids and Lewis bases listed below. Examples of Lewis bases include oxo acid ions (nitrate ions, perchlorate ions, etc.), metal oxo acid ions (permanganate ions etc.), halide ions, thiocyanate ions, cyanate ions, and the like. Examples of Lewis acids include metal ions such as alkali metal ions and alkaline earth metal ions; polyatomic ions such as ammonium ions; complex ions; and the like. Specific examples of inorganic salts include lithium salts such as lithium chloride, lithium bromide, lithium iodide, lithium nitrate, lithium perchlorate, and lithium thiocyanate; calcium salts such as calcium chloride, calcium bromide, calcium iodide, calcium nitrate, calcium perchlorate, and calcium thiocyanate; iron salts such as iron chloride, iron bromide, iron iodide, iron nitrate, iron perchlorate, and iron thiocyanate; aluminum salts such as aluminum chloride, aluminum bromide, aluminum iodide, aluminum nitrate, aluminum perchlorate, and aluminum thiocyanate; potassium salts such as potassium chloride, potassium bromide, potassium iodide, potassium nitrate, potassium perchlorate, and potassium thiocyanate; sodium salts such as sodium chloride, sodium bromide, sodium iodide, sodium nitrate, sodium perchlorate, and sodium thiocyanate; zinc salts such as zinc chloride, zinc bromide, zinc iodide, zinc nitrate, zinc perchlorate, and zinc thiocyanate; magnesium salts such as magnesium chloride, magnesium bromide, magnesium iodide, magnesium nitrate, magnesium perchlorate, and magnesium thiocyanate; barium salts such as barium chloride, barium bromide, barium iodide, barium nitrate, barium perchlorate, and barium thiocyanate; strontium salts such as strontium chloride, strontium bromide, strontium iodide, strontium nitrate, strontium perchlorate, and strontium thiocyanate; and the like.

The inorganic salt content may be 1.0 part by mass or more, 5.0 parts by mass or more, 9.0 parts by mass or more, 15 parts by mass or more, or 20.0 parts by mass or more, based on 100 parts by mass of the total amount of the protein. The inorganic salt content may be 40 parts by mass or less, 35 parts by mass or less, or 30 parts by mass or less, based on 100 parts by mass of the total amount of the protein.

The protein solution may further contain various additives, if necessary. Examples of additives include plasticizers, leveling agents, crosslinking agents, crystal nucleating agents, antioxidants, ultraviolet absorbers, colorants, fillers, and synthetic resins. The additive content may be 50 parts by mass or less based on 100 parts by mass of the total amount of the protein.

In the exposure step, the molded article precursor obtained, for example, in the above manner is exposed to an environment with a relative humidity of 80% or more (hereinafter also referred to as “the exposure environment”). The relative humidity in the present invention refers to a value obtained by converting a relative humidity measured by a hygrometer (e.g., 7542-00 Highest II Hygrometer with Thermometer, produced by Sato Keiryoki Mfg. Co., Ltd.) into a relative humidity at 25° C.

In terms of further improving the degree of crystallization of the molded article, the relative humidity of the exposure environment is preferably 81.0% or more, 81.5% or more, 82.0% or more, 82.5% or more, 83.0% or more, 83.5% or more, or 84.0% or more; and more preferably 85.0% or more, 90.0% or more, or 95.0% or more. Under these circumstances, it is preferable to adjust the relative humidity of the exposure environment so that the water content of the molded article precursor (molded article intermediate) placed in the exposure environment is 8.5 mass % or more, 10 mass % or more, 13 mass % or more, 15 mass % or more, 17 mass % or more, or 18 mass % or more, based on the total amount of the molded article intemiediate.

The temperature of the exposure environment is not particularly limited. For example, the temperature of the exposure environment may be 0° C. or more, 5° C. or more, 15° C. or more, 20° C. or more, or 25° C. or more, and may be, for example, 120° C. or less, 100° C. or less, 80° C. or less, 60° C. or less, or 40° C. or less.

The time during which the molded article precursor is exposed to the environment with a relative humidity of 80% or more is not particularly limited, and is suitably selected depending on the shape, size, thickness, etc., of the molded article precursor. For example, the time may be 10 seconds or more, 10 minutes or more, 1 hour or more, or 24 hours or more; and may be, for example, 336 hours or less or 168 hours or less.

The atmosphere of the exposure environment is not particularly limited, and may be an air atmosphere, for example. The pressure of the exposure environment is not particularly limited, and may be, for example, atmospheric pressure or increased pressure.

The production method according to the present embodiment, may further include the step of drying the molded article precursor (drying step) before the exposure step. This makes it possible to reduce the water content of the molded article precursor before the exposure step to zero or a value close to zero. As a result, the operation of adjusting the relative humidity of the exposure environment so that the water content of the molded article precursor placed in the exposure environment reaches a desired value based on the total amount of the molded article precursor (molded article intermediate) can be performed more easily than when the moisture content of the molded article precursor before the exposure step is unknown (when a drying step is not performed). The molded article may be dried after the exposure step. The drying before or after the exposure step may be, for example, vacuum drying, heat drying, or vacuum heat drying.

A molded article having a high degree of crystallization is obtained through the exposure step described above. Moreover, a molded article having a large crystal size is obtained through the exposure step. In other words, it can be said that the present embodiment is, in one aspect, a method for improving the degree of crystallization of a molded article containing a protein by exposing the molded article to an environment with a relative humidity of 80% or more. It can also be said that the present embodiment is a method for improving the crystal size of a molded article containing a protein by exposing the molded article to an environment with a relative humidity of 80% or more.

The method for improving the degree of crystallization of the molded article or the method for improving the crystal size of the molded article as described above, may further include the step of drying the molded article before or after the exposure step. By drying the molded article before the exposure step, the water content of the molded article before the exposure step can be reduced to zero or to a value close to zero, as in the method for producing the molded article described above. As a result, the relative humidity of the exposure environment can be easily adjusted.

The present embodiment is, in one aspect, a molded article obtained by the abovementioned production method, that is, a molded article containing a protein and having an exposure history to an environment with a relative humidity of 80% or more. When the obtained molded article is a film, the thickness of the film may be, for example, 3 to 1000 μm or 5 to 100 μm. When the obtained molded article is a fiber, the average diameter of the fiber may be, for example, 5 to 300 μm or 5 to 50 μm.

### EXAMPLES

The present invention is described in more detail below based on Examples; however, the present invention is not limited to the following Examples.

### Example 1

Films were produced using cocoons of natural silkworms (Bombyx mori) according to the procedure described by D. N. Rockwood et al. (Nature Protocols, vol. 6 [10] (2011)). The outline of the procedure is shown below.

First, the silkworm cocoons from which the contents had been removed were cut into small pieces and boiled in 0.02M sodium carbonate (Na₂CO₃) aqueous solution for 30 minutes. Thereafter, a step of washing the obtained silk with Milli-Q water for 20 minutes was repeated three times. Subsequently, the silk was drained and dried. The dried silk was immersed in 9.3M lithium bromide (LiBr) aqueous solution, and was dissolved at 60° C. over about 4 hours. The obtained solution was transferred to a dialysis membrane, and dialysis was carried out for about 72 hours. The solution after dialysis was centrifuged at 12700G at 4° C. for 20 minutes to remove impurities. After repeating this process several times, the solution supernatant (protein concentration: 7.4 mass %) was poured into a plate, and then dried. Silkworm films (films containing a silk protein) were obtained in this manner. The obtained silkworm films had a thickness of about 55 μm to 75 μm.

Separately, in order to create intended humid environments, saturated salt solutions were prepared using Milli-Q water and several types of salts. Table 1 shows the type of salt used and humid environments created using the saturated salt solutions (showing values described in JISB 7920).

TABLE 1

| | Type of salt | | | | | | | | | |
|---|---|---|---|---|---|---|---|---|---|---|
| | LiBr | LiCl | CH₃COOK | MgCl₂ | K₂CO₃ | NaBr | KI | NaCl | KCl | K₂SO₄ |
| Relative | 6.4 | 11.3 | 22.5 | 32.8 | 43.2 | 57.6 | 68.9 | 75.3 | 84.2 | 97.3 |
| humidity | | | | | | | | | | |
| (%) at | | | | | | | | | | |
| 25° C. | | | | | | | | | | |

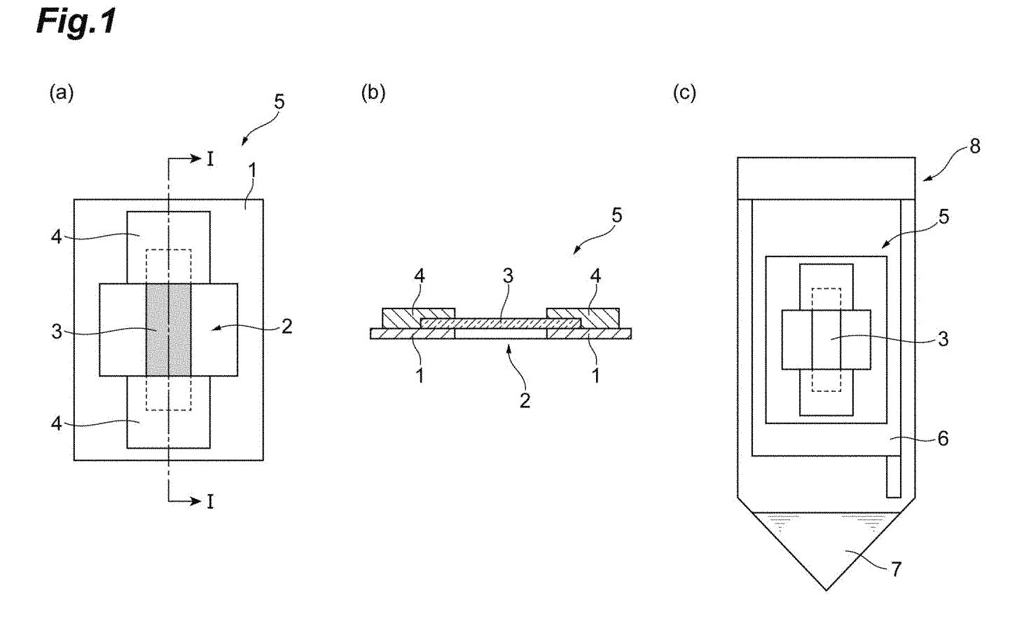

Next, the produced silkworm films were cut into a size of 12 mm×12 mm, and a plurality of films was obtained. Thereafter, each film was vacuum-dried at 40° C. for 24 hours. Subsequently, as shown in FIGS. 1(a) and (b) (FIG. 1 (b) is a cross-sectional view taken along line I-I of FIG. 1(a)), the film 3 after drying was placed in a window part 2 provided in the center of a support 1, and both ends of the film 3 were fixed to the support 1 by a fixing part 4, thereby producing a sample 5. The same number of samples 5 as the number of films was produced in the same manner. The produced samples 5 were each exposed to different saturated salt solution (humid) environments at 24.2° C. for about one week. Under this circumstance, as shown in FIG. 1(c), each sample 5 was placed in a syringe 6, and the syringe 6, together with a saturated salt solution 7, were placed in an airtight container 8 so that the film 3 was exposed to each humid environment with an air atmosphere without being immersed in the saturated salt solution 7. Aside from the abovementioned humid environments, a film immediately after vacuum-drying at 40° C. for 24 hours was placed in a syringe 6, and the syringe 6 was placed in an airtight container 8 filled with a drying agent (but not containing a saturated salt solution 7), thereby preparing an environment with a relative humidity of 0% (dry). A sample 5 different from those exposed to the abovementioned humid environments was exposed to this environment for about one week.

The samples 5 exposed to the different measurement environments in the abovementioned manner were brought into a large synchrotron radiation facility SPring-8 while being placed in the saturated salt solution environments, and wide-angle X-ray scattering measurements were performed. BL45XU was used for the measurements of wide-angle X-ray scattering (WAXD or WAXS).

The measurement conditions of the device are as follows.
Wavelength: 1.0 Å
Detector: Pilatus3 2M (produced by Rigaku Corporation)
Camera length: 244.84 mm
Exposure time: 10 sec
Beam center: [X] 727.63 mm, [Y] 864.79 mm

The humidity conditions of the wide-angle X-ray scattering measurements of the samples 5 were set as similar as possible to the settled saturated salt solution environments. Table 2 shows the corresponding relationship between the humidity during the exposure of each sample 5 to each saturated salt solution environment and the humidity during wide-angle X-ray scattering measurement.

TABLE 2

| | | | | | | | | | | | |
|---|---|---|---|---|---|---|---|---|---|---|---|
| Relative | dry | 6.4 | 11.3 | 22.5 | 32.8 | 43.2 | 57.6 | 68.9 | 75.3 | 84.2 | 97.3 |
| humidity | (0) | | | | | | | | | | |
| (%) during | | | | | | | | | | | |
| exposure | | | | | | | | | | | |
| Relative | 0.10 | 5.83 | 11.03 | 22.50 | 33.03 | 42.01 | 58.83 | 68.50 | 75.10 | 85.23 | 96.41 |
| humidity | | | | | | | | | | | |
| (%) during | | | | | | | | | | | |
| measurement | | | | | | | | | | | |

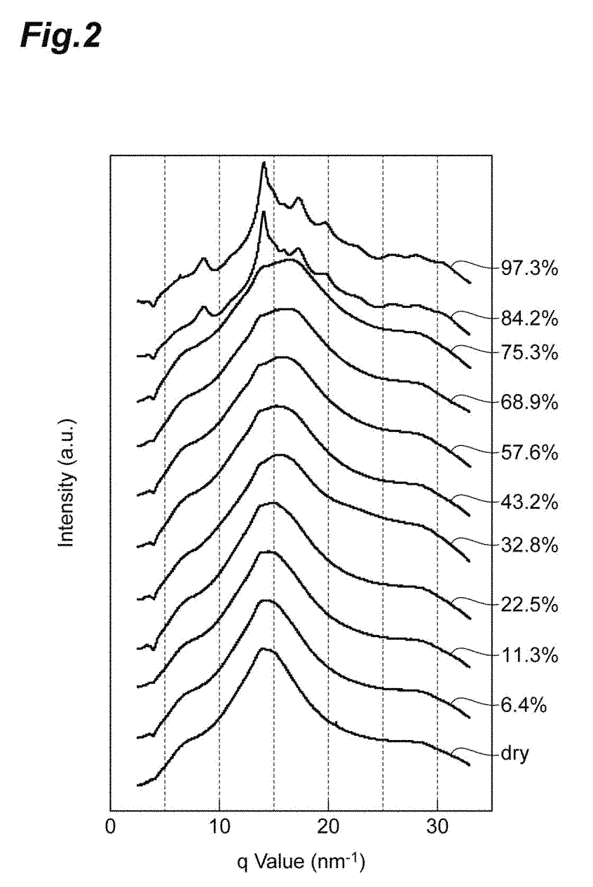

One-dimensional profiles of wide-angle X-ray scattering were created using data analyses software KaleidaGraph. FIG. 2 shows the obtained diffraction data. The degree of crystallization (%) was determined from the obtained diffraction data by the following formula (1):

Degree of crystallization (%)=[peak area showing crystalline region/(peak area showing crystalline region+peak area showing amorphous region)]×100 (1)

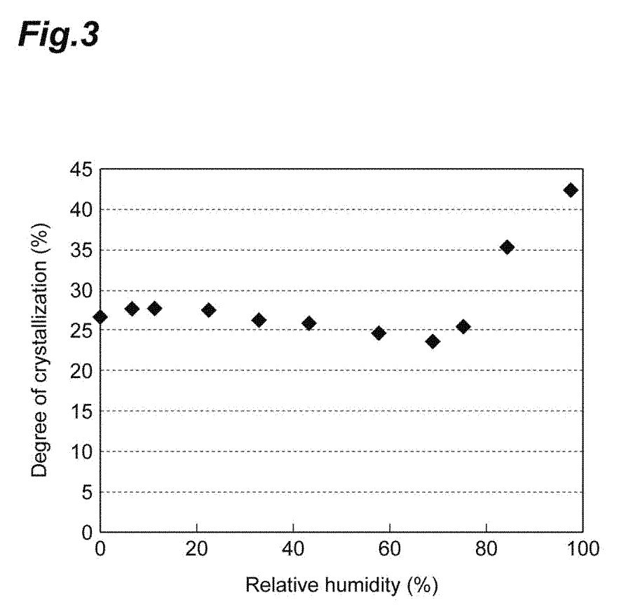

FIG. 3 shows the relationship between the relative humidity of the exposure environments and the degree of crystallization of the films.

As is clear from the results of FIGS. 2 and 3, it was confirmed that the degree of crystallization of the molded articles (films) containing a silk protein was improved by exposing them to environments with a relative humidity of 80% or more.

### Example 2

Next, films were produced in the following manner using a recombinant spider silk protein.

1. Production of Recombinant Spider Silk Protein (Recombinant Spider Silk Fibroin: PRT410)

### (Synthesis of Gene Encoding Spider Silk Protein, and Construction of Expression Vector)

Modified fibroin having the amino acid sequence represented by

SEQ ID NO: 1 (hereinafter also referred to as “PRT410”) was designed based on the base sequence and amino acid sequence of fibroin derived from Nephila clavipes (GenBank Accession Number: P46804.1, GI: 1174415).

The amino acid sequence represented by SEQ ID NO: 1 has an amino acid sequence with substitution, insertion, and deletion of amino acid residues in the amino acid sequence of fibroin derived from Nephila clavipes for the purpose of improving productivity, and the amino acid sequence represented by SEQ ID NO: 7 (tag sequence and hinge sequence) is further added to the N-terminal.

Next, a nucleic acid encoding PRT410 was synthesized. An NdeI site was added to the 5′-end of the nucleic acid, and an EcoRI site was added to the downstream of the stop codon. The nucleic acid was cloned into a cloning vector (pUC118). Thereafter, the nucleic acid was digested with restriction enzymes NdeI and EcoRI, and then recombined into a protein expression vector pET-22b(+). Thus, the expression vector was obtained.

Escherichia coli BLR(DE3) was transformed with the pET22b(+) expression vector containing the nucleic acid encoding PRT410. The transformed Escherichia coli was cultured in 2 mL of LB medium containing ampicillin for 15 hours. The culture solution was added to 100 mL of seed culture medium containing ampicillin (Table 3) so that the OD₆₀₀ was 0.005. The culture solution temperature was maintained at 30° C., and flask culture was performed (for about 15 hours) until the OD₆₀₀ reached 5, thereby obtaining a seed culture solution.

TABLE 3
Seed culture medium

| Reagent | Concentration (g/L) |
|---|---|
| Glucose | 5.0 |
| KH₂PO₄ | 4.0 |
| K₂HPO₄ | 9.3 |
| Yeast extract | 6.0 |
| Ampicillin | 0.1 |

The seed culture solution was added to a jar fermenter, to which 500 ml of production medium (Table 4 below) was added, so that the OD₆₀₀ was 0.05. The culture solution temperature was maintained at 37° C., and culture was performed while constantly controlling the pH at 6.9. Moreover, the dissolved oxygen concentration of the culture solution was maintained at 20% of the saturated dissolved oxygen concentration.

TABLE 4
Production medium

| Reagent | Concentration (g/L) |
|---|---|
| Glucose | 12.0 |
| KH₂PO₄ | 9.0 |
| MgSO₄•7H₂O | 2.4 |
| Yeast extract | 15 |
| FeSO₄•7H₂O | 0.04 |
| MnSO₄•5H₂O | 0.04 |
| CaCl₂•2H₂O | 0.04 |
| Adekanol (LG-295S, Adeka Corporation) | 0.1 (mL/L) |

Immediately after glucose in the production medium was completely consumed, a feed solution (glucose 455 g/1 L, yeast extract 120 g/1 L) was added at rate of 1 mL/min. The culture solution temperature was maintained at 37° C., and culture was performed while constantly controlling the pH at 6.9. Moreover, the dissolved oxygen concentration of the culture solution was maintained at 20% of the saturated dissolved oxygen concentration, and culture was performed for 20 hours. Thereafter, 1M isopropyl-β-thiogalactopyranoside (IPTG) was added to the culture solution to a final concentration of 1 mM, and the expression of PRT410 was induced. After the lapse of 20 hours since the adding of IPTG, the culture solution was centrifuged, and bacterial cells were collected. SDS-PAGE was carried out using bacterial cells prepared from the culture solution before and after IPTG was added, and the expression of PRT410 was confirmed by the appearance of a band of a size corresponding to PRT410 depending on IPTG addition.

(Purification of PRT410)

The bacterial cells which were collected 2 hours after the addition of IPTG were then washed with 20 mM Tris-HCl buffer (pH 7.4). The washed bacterial cells were suspended in 20 mM Tris-HCl buffer (pH 7.4) containing about 1 mM PMSF, and the cells were disrupted with a high-pressure homogenizer (produced by GEA Niro Soavi). The disrupted cells were centrifuged to obtain a precipitate. The obtained precipitate was washed with 20 mM Tris-HCl buffer (pH 7.4) to high purity. The precipitate after washing was suspended in 8M guanidine buffer (8M guanidinium hydrochloride, 10 mM sodium dihydrogen phosphate, 20 mM NaCl, 1 mM Tris-HCl, pH 7.0) to a concentration of 100 mg/mL, and was dissolved by stirring with a stirrer at 60° C. for 30 minutes. After dissolution, dialysis was carried out against water using a dialysis tube (Cellulose Tube 36/32, produced by Sanko Junyaku Co., Ltd.). White aggregated protein (PRT410) obtained after dialysis was collected by centrifugal separation, moisture was removed by a freeze dryer, and a freeze-dried powder was collected.

The degree of purification of PRT410 in the obtained freeze-dried powder was confirmed by image analysis of the results of polyacrylamide gel electrophoresis of the powder using TotalLab (Nonlinear Dynamics Ltd.). As a result, the degree of purification of PRT410 was about 85%.

2. Production of Spider Silk Protein Film (Spider Silk Fibroin Film)

(Preparation of Dope Solution)

18 g of the abovementioned recombinant spider silk fibroin (PRT410), 57 g of pure water, 24 g of Clynsolve P-7, and 1 g of glycerol were supplied in a high-pressure microreactor (model “MMJ-500,” produced by OM Labotech). The reactor was closed with a lid, and the spider silk fibroin was dissolved by heating at 100° C. for 40 minutes, thereby preparing a dope solution (protein ratio: 18 mass %).

(Film Cast Molding)

The prepared dope solution was cast-molded on the surface of a substrate using a coating machine (model “IMC-70F-B,” produced by Imoto Machinery Co., Ltd.) to form a wet film. The substrate used was a release film where a silicone compound was fixed to the surface of a polyethylene terephthalate film (PET) having a thickness of 75 μm (trade name “Purex,” produced by DuPont Teijin Films; 38 μm).

(Drying)

The molded wet film was dried by allowing it to stand at 60° C. for 2 minutes, and at 100° C. for 2 minutes. Then, the film was removed from the substrate. The thus-obtained spider silk fibroin film (film containing spider silk fibroin) had a thickness of about 16 μm.

Next, the produced spider silk fibroin film was cut into a size of 20 mm×20 mm to obtain three films. The three films were each exposed to different saturated salt solution (humid) environments at 25° C. for about one day in the same manner as in Example 1, except that the type of salt used was changed to NaCl, KCl, and K₂SO₄. Thereafter, the films were allowed to stand in a constant-temperature constant-humidity chamber (LHL-113, produced by Espec Corp.) under conditions at 20° C/165% for about 3 days.

The three films exposed to the different humid environments in the abovementioned manner were brought into a large synchrotron radiation facility SPring-8 while being placed in the saturated salt solution environments, and wide-angle X-ray scattering measurements were performed for each film in the same manner as in Example 1.

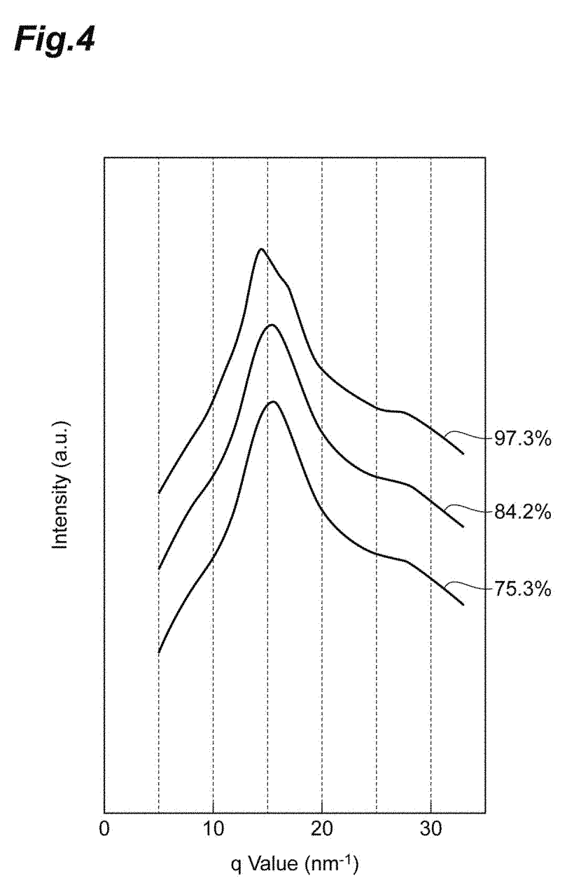

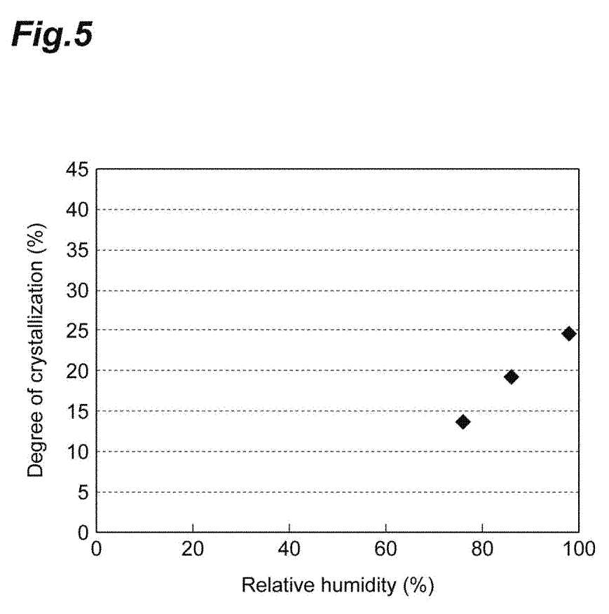

One-dimensional profiles of wide-angle X-ray scattering were created using data analyses software KaleidaGraph in the same manner as in Example 1. FIG. 4 shows the obtained diffraction data. Further, the degree of crystallization (%) was determined from the obtained diffraction data by the abovementioned formula (1). FIG. 5 shows the relationship between the relative humidity of the exposure environments and the degree of crystallization of the films.

As is clear from the results of FIGS. 4 and 5, it was confirmed that the degree of crystallization of the molded articles (films) containing a recombinant spider silk protein was improved by exposing them to environments with a relative humidity of 80% or more.

### REFERENCE SIGNS LIST

1: support, 2: window part, 3: film, 4: fixing part, 5: sample, 6: syringe, 7: saturated salt solution, 8: airtight container.

1. A method for producing a molded article, the method comprising exposing a molded article precursor comprising a protein to an environment with a relative humidity of 80% or more to obtain the molded article.

2. The method according to claim 1, further comprising drying the molded article precursor before exposing the molded article precursor to the environment.

3. The method according to claim 1, wherein the protein is a structural protein.

4. The method according to claim 1, wherein the protein is at least one selected from the group consisting of keratin, collagen, elastin, resilin, silk fibroin, and spider silk fibroin.

5. The method according to claim 1, wherein the protein is spider silk fibroin.

6. A molded article comprising a protein having an exposure history to an environment with a relative humidity of 80% or more.

7. The molded article according to claim 6, wherein the protein is a structural protein.

8. The molded article according to claim 6, wherein the protein is at least one selected from the group consisting of keratin, collagen, elastin, resilin, silk fibroin, and spider silk fibroin.

9. The molded article according to claim 6, wherein the protein is spider silk fibroin.

10. A method for improving a degree of crystallization of a molded article comprising a protein, the method comprising exposing the molded article to an environment with a relative humidity of 80% or more.

11. The method according to claim 10, further comprising drying the molded article before exposing the molded article to the environment.

12. The method according to claim 10, wherein the protein is a structural protein.

13. The method according to claim 10, wherein the protein is at least one selected from the group consisting of keratin, collagen, elastin, resilin, silk fibroin, and spider silk fibroin.

14. The method according to claim 10, wherein the protein is spider silk fibroin.


SEQUENCE LISTING (7 sequences)

SEQ ID NO: 1 - protein, 601 aa, Artificial Sequence
  PRT410 (CRY1_L_A5_giza_QQQ)
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 2 - protein, 559 aa, Artificial Sequence
  Recombinant spider silk protein Flag_92_short2
MHHHHHHHHHHSSGSSLEVLFQGPGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGGGPGGYGPGGAGPGGYGPGGSGPGGYGPGGSGPGGYGPGGSGPGGYGPGGSGPGGYGPGGSGPGGSGPGGYGPGGSGPGGSGPGGYGPGGSGPGGYGPGGSGPGGSGPGGYGPGGSGPGGSGPGGYGPGGSGPGGFGPGGFGPGGSGPGGYGPGGSGPGGAGPGGVGPGGFGPGGAGPGGAGPGGAGPGGAGPGGAGPGGAGPGGAGPGGAGPGGAGGAGGAGGAGGSGGAGGSGGTTIIEDLDITIDGADGPITISEELTISAYYPSSRVPDMVNGIMSAMQGSGFNYQMFGNMLSQYSSGSGTCNPNNVNVLMDALLAALHCLSNHGSSSFAPSPTPAAMSAYSNSVGRMFAY

SEQ ID NO: 3 - protein, 252 aa, Artificial Sequence
  Collagen-type4-Kai
MHHHHHHSSGSSKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEK

SEQ ID NO: 4 - protein, 310 aa, Artificial Sequence
  Resilin-Kai
MHHHHHHPEPPVNSYLPPSDSYGAPGQSGPGGRPSDSYGAPGGGNGGRPSDSYGAPGQGQGQGQGQGGYAGKPSDSYGAPGGGDGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASG

SEQ ID NO: 5 - protein, 282 aa, Artificial Sequence
  elastin short
MHHHHHHSSGSSLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVG

SEQ ID NO: 6 - protein, 468 aa, Artificial Sequence
  type I keratin 26
MSFRLSGVSRRLCSQAGTGRLTGGRTGFRAGNVCSGLGAGSSFSGPLGSVSSKGSFSHGGGGLGSGVCTGFLENEHGLLPGNEKVTLQNLNDRLASYLDHVCTLEEANADLEQKIKGWYEKYGPGSGRQLAHDYSKYFSVTEDLKRQIISVTTCNASIVLQNENARLTADDFRLKCENELALHQSVEADINGLHRVMDELTLCTSDLEMQCEALSEELTYLKKNHQEEMKVMQGAARGNVNVEINAAPGVDLTVLLNNMRAEYEDLAEQNHEDAEAWFSEKSTSLHQQISDDAGAAMAARNELMELKRNLQTLEIELQSLLAMKHSYECSLAETESNYCHQLQQIQEQIGAMEDQLQQIRMETEGQKLEHERLLDVKIFLEKEIEMYCKLIDGEGRKSKSTCYKSEGRGPKNSENQVKDSKEEAVVKTVVGELDQLGSVLSLRVHSVEEKSSKISNITMEQRLPSKVP

SEQ ID NO: 7 - protein, 12 aa, Artificial Sequence
  His Tag
MHHHHHHSSGSS